(19) United States
(12) Patent Application Publication
(10) Pub. No.: US 2022/0202470 A1
(43) Pub. Date: Jun. 30, 2022

(54) ELECTROSURGICAL INSTRUMENT SYSTEM WITH PARASITIC ENERGY LOSS MONITOR

(71) Applicant: Ethicon LLC, Guaynabo, PR (US)

(72) Inventor: Frederick E. Shelton, IV, Hillsboro, OH (US)

(21) Appl. No.: 17/136,139

(22) Filed: Dec. 29, 2020

(51) Int. Cl. A61B 18/12; A61B 18/16; A61B 34/37

(52) U.S. Cl. CPC A61B 18/1206; A61B 2018/00077; A61B 34/37; A61B 18/16

(57) ABSTRACT

A method of performing an electrosurgical procedure includes activating an electrode of a surgical instrument by applying an output power signal with a first energy output profile from a generator to the electrode. An induced electrical parameter of a conductive component is monitored via one or more sensors, the induced electrical parameter being associated with a predetermined electrical parameter threshold. The induced electrical parameter includes a parasitic energy loss. When the induced electrical parameter measured from a conductive component of the surgical instrument meets or exceeds the predetermined electrical parameter threshold during the operation, the output power signal of the generator is adjusted from a first energy output profile to a second energy output profile. The adjustment is operable to reduce the induced electrical parameter measured from the conductive component of the surgical instrument; and to reduce the parasitic energy loss without ceasing delivery of energy to the electrode.

### BACKGROUND

A variety of ultrasonic surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned, the disclosure of which is incorporated by reference herein, in its entirety; U.S. Pub. No. 2007/0191713, entitled “Ultrasonic Device for Cutting and Coagulating,” published Aug. 16, 2007, now abandoned, the disclosure of which is incorporated by reference herein, in its entirety; and U.S. Pub. No. 2008/0200940, entitled “Ultrasonic Device for Cutting and Coagulating,” published Aug. 21, 2008, now abandoned, the disclosure of which is incorporated by reference herein, in its entirety.

Some instruments are operable to seal tissue by applying radiofrequency (RF) electrosurgical energy to the tissue. Examples of such devices and related concepts are disclosed in U.S. Pat. No. 7,354,440, entitled “Electrosurgical Instrument and Method of Use,” issued Apr. 8, 2008, the disclosure of which is incorporated by reference herein, in its entirety; U.S. Pat. No. 7,381,209, entitled “Electrosurgical Instrument,” issued Jun. 3, 2008, the disclosure of which is incorporated by reference herein, in its entirety.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein, in its entirety; and U.S. Pat. No. 8,663,220, entitled “Ultrasonic Electrosurgical Instruments,” issued Mar. 4, 2014, the disclosure of which is incorporated by reference herein, in its entirety.

In some scenarios, it may be preferable to have surgical instruments grasped and manipulated directly by the hand or hands of one or more human operators. In addition, or as an alternative, it may be preferable to have surgical instruments controlled via a robotic surgical system. Examples of robotic surgical systems and associated instrumentation are disclosed in U.S. Pat. No. 10,624,709, entitled “Robotic Surgical Tool with Manual Release Lever,” published on May 2, 2019, the disclosure of which is incorporated by reference herein, in its entirety; U.S. Pat. No. 9,314,308, entitled “Robotic Ultrasonic Surgical Device With Articulating End Effector,” issued on Apr. 19, 2016, the disclosure of which is incorporated by reference herein, in its entirety; U.S. Pat. No. 9,125,662, entitled “Multi-Axis Articulating and Rotating Surgical Tools,” issued Sep. 8, 2015, the disclosure of which is incorporated by reference herein, in its entirety; U.S. Pat. No. 8,820,605, entitled “Robotically-Controlled Surgical Instruments,” issued Sep. 2, 2014, the disclosure of which is incorporated by reference herein, in its entirety; U.S. Pub. No. 2019/0201077, entitled “Interruption of Energy Due to Inadvertent Capacitive Coupling,” published Jul. 4, 2019, the disclosure of which is incorporated by reference herein, in its entirety; U.S. Pub. No. 2012/0292367, entitled “Robotically-Controlled End Effector,” published on Nov. 11, 2012, now abandoned, the disclosure of which is incorporated by reference herein, in its entirety; and U.S. patent application Ser. No. 16/556,661, entitled “Ultrasonic Surgical Instrument with a Multi-Planar Articulating Shaft Assembly,” filed on Aug. 30, 2019, the disclosure of which is incorporated by reference herein, in its entirety.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a schematic view of an example of a robotic surgical system;

FIG. 2 depicts a schematic view of an example of a robotic surgical system being used in relation to a patient;

FIG. 3 depicts a schematic view of examples of components that may be incorporated into a surgical instrument;

FIG. 4 depicts a side elevation view of an example of a handheld surgical instrument;

FIG. 5 depicts a perspective view of an example of an end effector that is operable to apply ultrasonic energy to tissue;

FIG. 6 depicts a perspective view of an example of an end effector that is operable to apply bipolar RF energy to tissue;

FIG. 7 depicts a schematic view of an example of a surgical instrument that is operable to apply monopolar RF energy to tissue;

FIG. 8 depicts a perspective view of an example of an articulation section that may be incorporated into a shaft assembly of a surgical instrument;

FIG. 9 depicts a side elevation view of a portion of a shaft assembly that may be incorporated into a surgical instrument, with housing components of the shaft being shown in cross-section to reveal internal components of the shaft;

FIG. 10 depicts a cross-sectional end view of another shaft assembly that may be incorporated into a surgical instrument;

FIG. 11 depicts a schematic view of a portion of another shaft assembly that may be incorporated into a surgical instrument;

FIG. 12 depicts a perspective view of an example of a surgical instrument that may be incorporated into the robotic surgical system of FIG. 1;

FIG. 13 depicts a top plan view of an interface drive assembly of the instrument of FIG. 12;

FIG. 14 depicts a cross-sectional side view of an articulation section of a shaft assembly of the instrument of FIG. 12;

FIG. 15 depicts a perspective view of another example of a handheld surgical instrument, with a modular shaft assembly separated from a handle assembly;

FIG. 16 depicts a schematic view of another example of a surgical instrument that is operable to apply monopolar RF energy to tissue; and

FIG. 17 depicts a flowchart of an exemplary method of monitoring the energy loss of a surgical instrument that is operable to apply RF energy to tissue.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” “bottom,” “above,” and “below,” are used with respect to the examples and associated figures and are not intended to unnecessarily limit the invention described herein.

### I. EXAMPLE OF A ROBOTIC SURGICAL SYSTEM

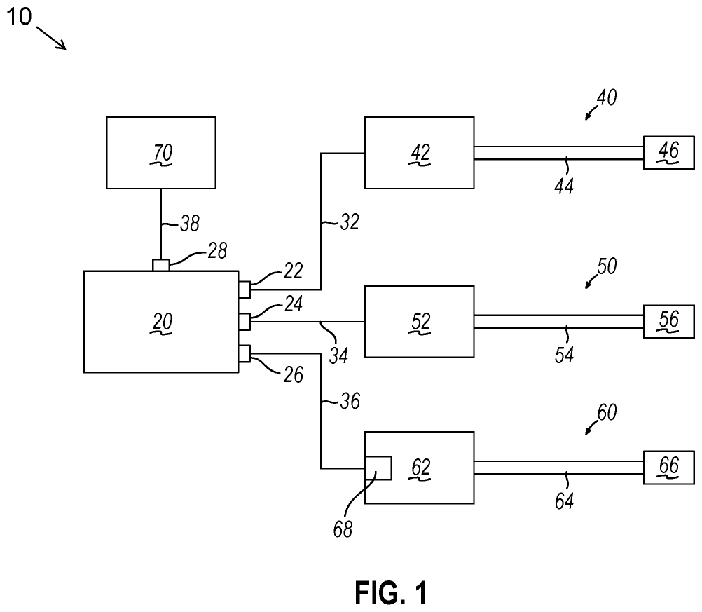

As noted above, in some surgical procedures, it may be desirable to utilize a robotically controlled surgical system. Such a robotically controlled surgical system may include one or more surgical instruments that are controlled and driven robotically via one or more users that are either in the same operating room or remote from the operating room. FIG. 1 illustrates on example of various components that may be incorporated into a robotic surgical system (10). System (10) of this example includes a console (20), a monopolar RF electrosurgical instrument (40), a bipolar RF electrosurgical instrument (50), and an ultrasonic surgical instrument (60). While FIG. 1 shows all three instruments (40, 50, 60) coupled with console (20) at the same time, there may be usage scenarios where only one or two of instruments (40, 50, 60) coupled with console (20) at the same time. In addition, there may be usage scenarios where various other instruments are coupled with console (20) in addition, or as an alternative to, one or more of instruments (40, 50, 60) being coupled with console (20).

Monopolar RF electrosurgical instrument (40) of the present example includes a body (42), a shaft (44) extending distally from body (42), and an end effector (46) at the distal end of shaft (44). Body (42) is configured to couple with a robotic arm (not shown in FIG. 1) of system (10), such that the robotic arm is operable to position and orient monopolar RF electrosurgical instrument (40) in relation to a patient. In versions where monopolar RF electrosurgical instrument (40) includes one or more mechanically driven components (e.g., jaws at end effector (46), articulating sections of shaft (44), rotating sections of shaft (44), etc.), body (42) may include various components that are operable to convert one or more mechanical drive inputs from the robotic arm into motion of the one or more mechanically driven components of monopolar RF electrosurgical instrument (40).

As also shown in FIG. 1, body (42) is coupled with a corresponding port (22) of console (20) via a cable (32). Console (20) is operable to provide electrical power to monopolar RF electrosurgical instrument (40) via port (22) and cable (32). In some versions, port (22) is dedicated to driving monopolar RF electrosurgical instruments like monopolar RF electrosurgical instrument (40). In some other versions, port (22) is operable to drive various kinds of instruments (e.g., including instruments (50, 60), etc.). In some such versions, console (20) is operable to automatically detect the kind of instrument (40, 50, 60) that is coupled with port (22) and adjust the power profile to port (22) accordingly. In addition, or in the alternative, console (20) may adjust the power profile to port (22) based on a selection made by an operator via console (20), manually identifying the kind of instrument (40, 50, 60) that is coupled with port (22).

Shaft (44) is operable to support end effector (46) and provides one or more wires or other paths for electrical communication between base (42) and end effector (46). Shaft (44) is thus operable to transmit electrical power from console (20) to end effector (46). Shaft (44) may also include various kinds of mechanically movable components, including but not limited to rotating segments, articulating sections, and/or other kinds of mechanically movable components as will be apparent to those skilled in the art in view of the teachings herein.

End effector (46) of the present example includes an electrode that is operable to apply monopolar RF energy to tissue. Such an electrode may be incorporated into a sharp blade, a needle, a flat surface, some other atraumatic structure, or any other suitable kind of structure as will be apparent to those skilled in the art in view of the teachings herein. End effector (46) may also include various other kinds of components, including but not limited to grasping jaws, etc.

System (10) of this example further includes a ground pad (70) that is coupled with a corresponding port (28) of console (20) via a cable (38). In some versions, ground pad (70) is incorporated into a patch or other structure that is adhered to the skin of the patient (e.g., on the thigh of the patient). In some other versions, ground pad (70) is placed under the patient (e.g., between the patient and the operating table). In either case, ground pad (70) may serve as a return path for monopolar RF energy that is applied to the patient via end effector (46). In some versions, port (28) is a dedicated ground return port. In some other versions, port (28) is a multi-purpose port that is either automatically designated as a ground return port upon console (20) detecting the coupling of ground pad (70) with port (28) or manually designated as a ground return port via an operator using a user input feature of console (20).

Bipolar RF electrosurgical instrument (50) of the present example includes a body (52), a shaft (54) extending distally from body (52), and an end effector (56) at the distal end of shaft (54). Each of these components (52, 54, 56) may be configured and operable in accordance with the above description of corresponding components (42, 44, 46) of monopolar RF electrosurgical instrument (50), except that end effector (56) of this example is operable to apply bipolar RF energy to tissue. Thus, end effector (56) includes at least two electrodes, with those two electrodes being configured to cooperate with each other to apply bipolar RF energy to tissue. Bipolar RF electrosurgical instrument (50) is coupled with console (20) via a cable (34), which is further coupled with a port (24) of console (20). Port (24) may be dedicated to powering bipolar RF electrosurgical instruments. Alternatively, port (24) or may be a multi-purpose port whose output is determined based on either automatic detection of bipolar RF electrosurgical instrument (50) or operator selection via a user input feature of console (20).

Ultrasonic surgical instrument (60) of the present example includes a body (62), a shaft (64) extending distally from body (62), and an end effector (66) at the distal end of shaft (64). Each of these components (62, 64, 66) may be configured and operable in accordance with the above description of corresponding components (42, 44, 46) of monopolar RF electrosurgical instrument (50), except that end effector (66) of this example is operable to apply ultrasonic energy to tissue. Thus, end effector (66) includes an ultrasonic blade or other ultrasonically vibrating element. In addition, base (62) includes an ultrasonic transducer (68) that is operable to generate ultrasonic vibrations in response to electrical power, while shaft (64) includes an acoustic waveguide that is operable to communicate the ultrasonic vibrations from transducer (68) to end effector (66).

Ultrasonic surgical instrument (60) is coupled with console (20) via a cable (36), which is further coupled with a port (26) of console (20). Port (26) may be dedicated to powering ultrasonic electrosurgical instruments. Alternatively, port (26) or may be a multi-purpose port whose output is determined based on either automatic detection of ultrasonic instrument (60) or operator selection via a user input feature of console (20).

While FIG. 1 shows monopolar RF, bipolar RF, and ultrasonic capabilities being provided via three separate, dedicated instruments (40, 50, 60), some versions may include an instrument that is operable to apply two or more of monopolar RF, bipolar RF, or ultrasonic energy to tissue. In other words, two or more of such energy modalities may be incorporated into a single instrument. Examples of how such different modalities may be integrated into a single instrument are described in U.S. Pub. No. 2017/0202591, entitled “Modular Battery Powered Handheld Surgical Instrument with Selective Application of Energy Based on Tissue Characterization,” published Jul. 20, 2017, the disclosure of which is incorporated by reference herein, in its entirety. Other examples will be apparent to those skilled in the art in view of the teachings herein.

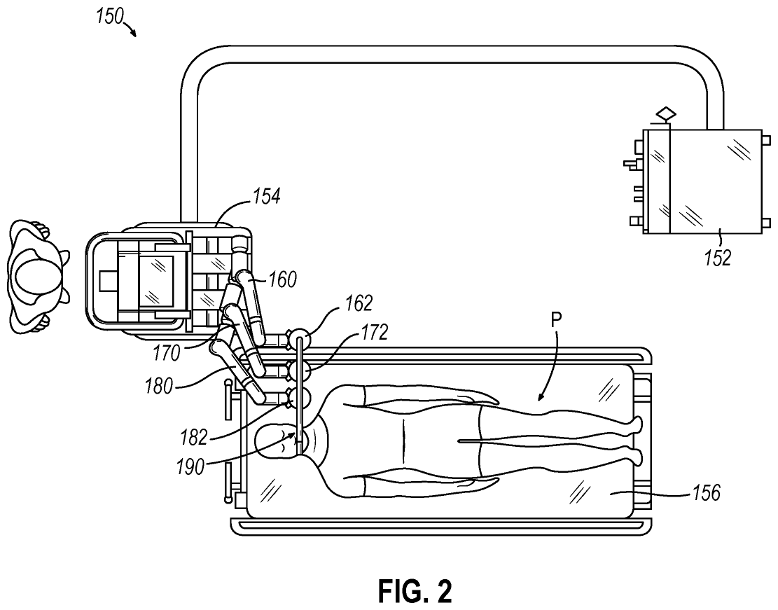

FIG. 2 shows an example of a robotic surgical system (150) in relation to a patient (P) on a table (156). System (150) of this example includes a control console (152) and a drive console (154). Console (152) is operable to receive user inputs from an operator; while drive console (154) is operable to convert those user inputs into motion of a set of robotic arms (160, 170, 180). In some versions, consoles (152, 154) collectively form an equivalent to console (20) described above. While consoles (152, 154) are shown as separate units in this example, consoles (152, 154) may in fact be combined as a single unit in some other examples.

Robotic arms (160, 170, 180) extend from drive console (154) in this example. In some other versions, robotic arms (160, 170, 180) are integrated into table (156) or some other structure. Each robotic arm (160, 170, 180) has a corresponding drive interface (162, 172, 182). In this example, three drive interfaces (162, 172, 182) are coupled with one single instrument assembly (190). In some other scenarios, each drive interface (162, 172, 182) is coupled with a separate respective instrument. By way of example only, a drive interface (162, 172, 182) may couple with a body of an instrument, like bodies (42, 52, 62) of instruments (40, 50, 60) described above. In any case, robotic arms (160, 170, 180) may be operable to move instrument (40, 50, 60, 190) in relation to the patient (P) and actuate any mechanically driven components of instrument (40, 50, 60, 190). Robotic arms (160, 170, 180) may also include features that provide a pathway for communication of electrical power to instrument (40, 50, 60, 190). For instance, cables (32, 34, 36) may be at least partially integrated into robotic arms (160, 170, 180). In some other versions, robotic arms (160, 170, 180) may include features to secure but not necessarily integrate cables (32, 34, 36). As yet another variation, cables (32, 34, 36) may simply stay separate from robotic arms (160, 170, 180). Other suitable features and arrangements that may be used to form robotic surgical systems (10, 150) will be apparent to those skilled in the art in view of the teachings herein.

In robotic surgical systems like robotic surgical systems (10, 150), each port (22, 24, 26, 28) may have a plurality of electrical features providing inputs and outputs between console (20, 152) and robotic arms (160, 170, 180) and/or instruments (40, 50, 60, 190). These electrical features may include sockets, pins, contacts, or various other features that are in close proximity with each other. In some scenarios, this proximity may provide a risk of power or signals undesirably crossing from one electrical feature to another electrical feature, which may cause equipment failure, equipment damage, sensor errors, and/or other undesirable results. In addition, or in the alternative, this proximity may provide a risk of generating electrical potentials between proximate components or creating capacitive couplings between electrical features. Such capacitive coupling may provide undesirable results such as power reductions, signal reductions, signal interference, patient injuries, and/or other undesirable results. It may therefore be desirable to provide features to prevent or otherwise address such occurrences at ports (22, 24, 26, 28).

Similarly, each robotic arm (160, 170, 180), each cable (32, 34, 36, 38), and/or each instrument (40, 50, 60, 190) may include a plurality of wires, traces in rigid or flexible circuits, and other electrical features that are in close proximity with each other. Such electrical features may also be in close proximity with other components that are not intended to provide pathways for electrical communication but are nevertheless formed of an electrically conductive material. Such electrically conductive mechanical features may include moving components (e.g., drive cables, drive bands, gears, etc.) or stationary components (e.g., chassis or frame members, etc.). This proximity may provide a risk of power or signals undesirably crossing from one electrical feature to another electrical feature and/or from one electrical feature to an electrically conductive mechanical feature, which may cause equipment failure, equipment damage, sensor errors, and/or other undesirable results. In addition, or in the alternative, this proximity may provide a risk of generating electrical potentials between proximate components or creating capacitive couplings between electrical features and/or between an electrical feature and an electrically conductive mechanical feature. Such capacitive coupling may provide undesirable results such as power reductions, signal reductions, signal interference, patient injuries, and/or other undesirable results. It may therefore be desirable to provide features to prevent or otherwise address such occurrences within robotic arms (160, 170, 180), within cables (32, 34, 36, 38), and/or within instruments (40, 50, 60, 190).

### II. EXAMPLE OF HANDHELD SURGICAL INSTRUMENT

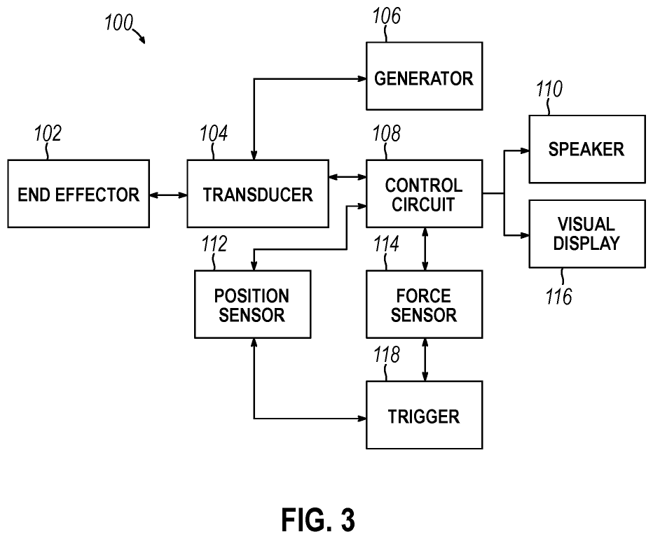

In some procedures, an operator may prefer to use a handheld surgical instrument in addition to, or in lieu of, using a robotic surgical system (10, 150). FIG. 3 illustrates an example of various components that may be integrated into a handheld surgical instrument (100). In addition to the following teachings, instrument (200) may be constructed and operable in accordance with at least some of the teachings of U.S. Pub. No. 2017/0202608, entitled “Modular Battery Powered Handheld Surgical Instrument Containing Elongated Multi-Layered Shaft,” published Jul. 20, 2017, the disclosure of which is incorporated by reference herein, in its entirety; and/or various other references cited herein. Instrument (100) of this example includes an end effector (102), an ultrasonic transducer (104), a power generator (106), a control circuit (108), a speaker (110), a position sensor (112), a force sensor (114), a visual display (116), and a trigger (118). In some versions, end effector (102) is disposed at a distal end of a shaft (not shown in FIG. 3), while the other components (104, 106, 108, 110, 112, 114, 116, 118) are incorporated into a handle assembly (not shown in FIG. 3) at the proximal end of the shaft. Some variations may also provide some of components (104, 106, 108, 110, 112, 114, 116, 118) in a separate piece of capital equipment. For instance, power generator (106), speaker (110), and/or visual display (116) may be incorporated into a separate piece of capital equipment that is coupled with instrument (100).

End effector (102) may be configured and operable like end effectors (46, 56, 66) described above, such that end effector (102) may be operable to apply monopolar RF energy, bipolar RF energy, or ultrasonic energy to tissue. Transducer (104) may be configured and operable like transducer (68). Generator (106) may be operable to provide electrical power as needed to drive transducer (68) and/or to provide RF energy via end effector (102). In versions where generator (106) is integrated into a handle assembly of instrument (106), generator (106) may comprise one or more battery cells, etc. Control circuit (108) may include one or more microprocessors and/or various other circuitry components that may be configured to provide signal processing and other electronic aspects of operability of instrument (100). Position sensor (112) may be configured to sense the position and/or orientation of instrument (102). In some versions, control circuit (108) is configured to vary the operability of instrument (102) based on data from position sensor (112). Force sensor (114) is operable to sense one or more force parameters associated with usage of instrument (100). Such force parameters may include force being applied to instrument (100) by the operator, force applied to tissue by end effector (102), or other force parameters as will be apparent to those skilled in the art in view of the teachings herein. In some versions, control circuit (108) is configured to vary the operability of instrument (102) based on data from force sensor (114). In some versions, one or both of sensors (112, 114) may be incorporated into end effector (102). In addition, or in the alternative, one or both of sensors (112, 114) may be incorporated into a shaft assembly (not shown) of instrument (100). Variations of instrument (100) may also incorporate various other kinds of sensors (e.g., in addition to or in lieu of sensors (112, 114) in end effector (102), in the shaft assembly, and/or elsewhere within instrument (100).

Trigger (118) is operable to control an aspect of operation of end effector (102), such as movement of a pivoting jaw, translation of a cutting blade, etc. Speaker (110) and visual display (116) are operable to provide audible and visual feedback to the operator relating to operation of instrument (100). The above-described components (102, 104, 106, 108, 110, 112, 114, 116, 118) of instrument (100) are illustrative examples, such that components (102, 104, 106, 108, 110, 112, 114, 116, 118) may be varied, substituted, supplemented, or omitted as desired.

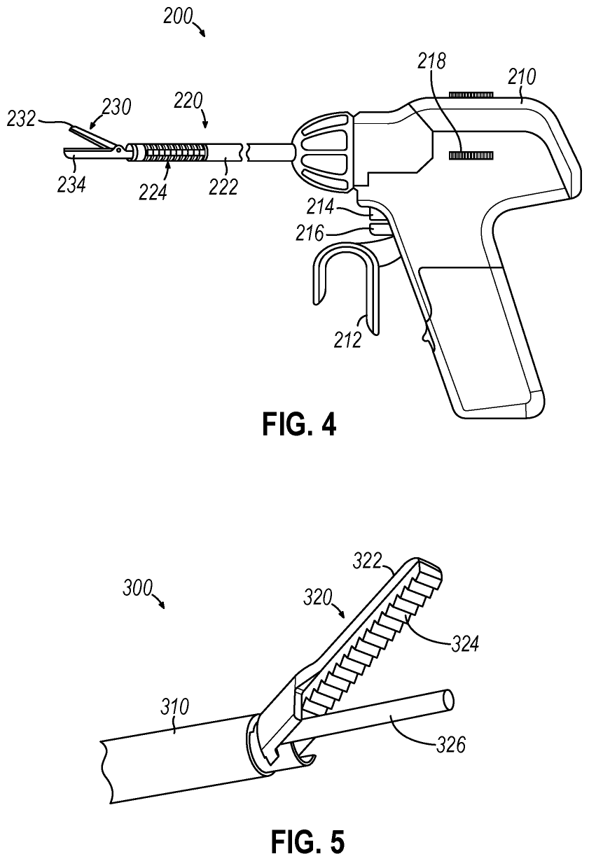

FIG. 4 shows an example of a form that instrument (100) may take. In particular, FIG. 4 shows a handheld instrument (200). In addition to the following teachings, instrument (200) may be constructed and operable in accordance with at least some of the teachings of U.S. Pub. No. 2017/0202591, the disclosure of which is incorporated by reference herein, in its entirety; and/or various other references cited herein. In the present example, instrument (200) includes a handle assembly (210), a shaft assembly (220), and an end effector (230). Handle assembly (210) includes a pivoting trigger (212), a first trigger button (214), a second trigger button (216), and an articulation control (218). Shaft assembly (220) includes a rigid shaft portion (222) and an articulation section (224). End effector (230) is distal to articulation section (224) and includes an upper jaw (232) and a lower jaw (234).

By way of example only, handle assembly (210) may include one or more of the above-described components (104, 106, 108, 110, 112, 114, 116, 118). Trigger (212) may be operable to drive upper jaw (232) to pivot toward lower jaw (234) (e.g., to grasp tissue between haws (232, 234)). Trigger buttons (214, 216) may be operable to activate delivery of energy (e.g., RF energy and/or ultrasonic energy) via end effector (230). Articulation control (218) is operable to drive deflection of shaft assembly (220) at articulation section (224), thereby driving lateral deflection of end effector (230) away from or toward the central longitudinal axis defined by rigid shaft portion (222). End effector (230) may include one or more electrodes that is/are operable to apply monopolar and/or bipolar RF energy to tissue. In addition, or in the alternative, end effector (230) may include an ultrasonic blade that is operable to apply ultrasonic energy to tissue. In some versions, end effector (230) is operable to apply two or more of monopolar RF energy, bipolar RF energy, or ultrasonic energy to tissue. Other suitable features and functionalities that may be incorporated into end effector (230) will be apparent to those skilled in the art in view of the teachings herein.

Instruments (150, 200) may include a plurality of wires, traces in rigid or flexible circuits, and other electrical features that are in close proximity with each other. Such electrical features may be located within handle assembly (210), within shaft assembly (220), and/or in end effector (230). Such electrical features may also be in close proximity with other components that are not intended to provide pathways for electrical communication but are nevertheless formed of an electrically conductive material. Such electrically conductive mechanical features may include moving components (e.g., drive cables, drive bands, gears, etc.) or stationary components (e.g., chassis or frame members, etc.). This proximity may provide a risk of power or signals undesirably crossing from one electrical feature to another electrical feature and/or from one electrical feature to an electrically conductive mechanical feature, which may cause equipment failure, equipment damage, sensor errors, patient injuries, and/or other undesirable results. In addition, or in the alternative, this proximity may provide a risk of generating electrical potentials between proximate components or creating capacitive couplings between electrical features and/or between an electrical feature and an electrically conductive mechanical feature. Such capacitive coupling may provide undesirable results such as power reductions, signal reductions, signal interference, and/or other undesirable results. It may therefore be desirable to provide features to prevent or otherwise address such occurrences within instruments (150, 200).

### III. FURTHER EXAMPLES OF SURGICAL INSTRUMENT COMPONENTS

The following description relates to examples of different features that may be incorporated into any of the various instruments (40, 50, 60, 100, 190, 200) described above. While these examples are provided separate from each other, the features described in any of the following examples may be combined with the features described in other examples described below. Thus, the below-described features may be combined in various permutations as will be apparent to those skilled in the art in view of the teachings herein. Similarly, various ways in which the below-described features may be incorporated into any of the various instruments (40, 50, 60, 100, 190, 200) described above will be apparent to those skilled in the art in view of the teachings herein. The below-described features may be incorporated into robotically controlled surgical instruments (40, 50, 60, 190) and/or handheld surgical instruments (100, 200).

A. Example of Ultrasonic End Effector

FIG. 5 shows a portion of an example of an ultrasonic instrument (300), including a shaft assembly (310) and an end effector (320). End effector (320) includes an upper jaw (322) and an ultrasonic blade (326). Upper jaw (322) is operable to pivot toward ultrasonic blade (326) to thereby compress tissue between a clamp pad (324) of upper jaw (322) and ultrasonic blade (326). When ultrasonic blade (326) is activated with ultrasonic vibrations, ultrasonic blade (326) may sever and seal tissue compressed against clamp pad (324). By way of example only, end effectors (66, 102, 230) may be configured and operable similar to end effector (320).

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (300), such risks may occur with respect to an acoustic waveguide in shaft assembly (310) leading to ultrasonic blade (326), as the acoustic waveguide may be formed of an electrically conductive material. In addition, instrument (300) may include one or more sensors in shaft assembly (310) and/or end effector (320); and may also include one or more electrodes and/or other electrical features in end effector (320). Other components of instrument (350) that may present the above-described risks will be apparent to those skilled in the art in view of the teachings herein.

B. Example of Bipolar RF End Effector

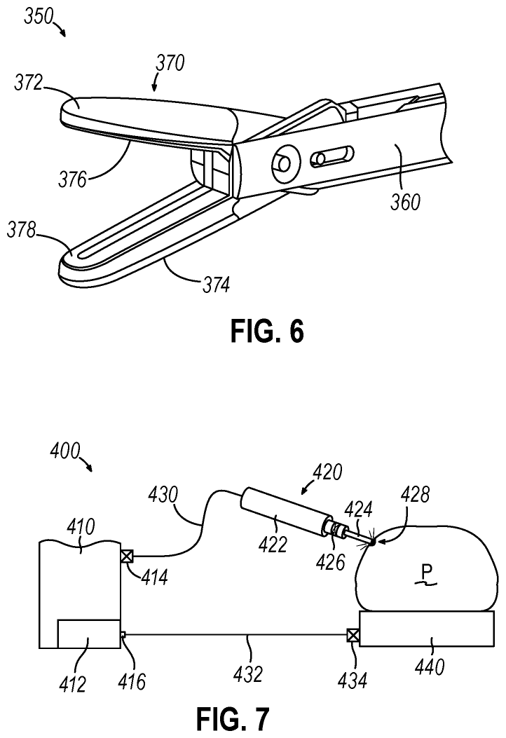

FIG. 6 shows a portion of an example of a bipolar RF instrument (350), including a shaft assembly (360) and an end effector (370). End effector (370) includes an upper jaw (372) and a lower jaw (374). Jaws (372, 374) are pivotable toward and away from each other. Upper jaw (372) includes a first electrode surface (376) while lower jaw (374) includes a second electrode surface (378). When tissue is compressed between jaws (372, 374), electrode surfaces (376, 378) may be activated with opposing polarities to thereby apply bipolar RF energy to the tissue. This bipolar RF energy may seal the compressed tissue. In some versions, end effector (370) further includes a translating knife member (not show) that is operable to sever tissue that is compressed between jaws (372, 374). Some variations of end effector (370) may also be operable to cooperate with a ground pad (e.g., ground pad (70)) to apply monopolar RF energy to tissue, such as by only activating one electrode surface (376, 378) or by activating both electrode surfaces (376, 378) at a single polarity. By way of example only, end effectors (64, 102, 230) may be configured and operable similar to end effector (370).

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (350), such risks may occur with respect to electrode surface (376, 378) and the wires or other electrical features that extend along shaft assembly (360) to reach electrode surfaces (376, 378). In addition, instrument (350) may include one or more sensors in shaft assembly (360) and/or end effector (370); and may also include one or more electrodes and/or other electrical features in end effector (370). Other components of instrument (350) that may present the above-described risks will be apparent to those skilled in the art in view of the teachings herein.

C. Example of Monopolar Surgical Instrument Features

FIG. 7 shows an example of a monopolar RF energy delivery system (400) that includes a power generator (410), a delivery instrument (420), and a ground pad assembly (440). In addition to the following teachings, instrument (420) may be constructed and operable in accordance with at least some of the teachings of U.S. Pub. No. 2019/0201077, the disclosure of which is incorporated by reference herein, in its entirety; and/or various other references cited herein. Power generator (410) may be operable to deliver monopolar RF energy to instrument (420) via a cable (430), which is coupled with power generator (410) via a port (414). In some versions, port (414) includes an integral sensor. By way of example only, such a sensor in port (414) may be configured to monitor whether excess or inductive energy is radiating from power generator (410) and/or other characteristics of energy being delivered from power generator (410) via port (414). Instrument (420) includes a body (422), a shaft (424), a sensor (426), and a distal electrode (428) that is configured to contact a patient (P) and thereby apply monopolar RF energy to the patient (P). By way of example only, sensor (426) may be configured to monitor whether excess or inductive energy is radiating from instrument (420). Based on signals from sensor (426), a control module in power generator (410) may passively throttle the ground return from ground pad assembly (440) based on data from sensor (426).

In some versions, ground pad assembly (440) comprises one or more resistive continuity ground pads that provide direct contact between the skin of the patient (P) and one or more metallic components of the ground pad. In some other versions, ground pad assembly (440) comprises a capacitive coupling ground pad that includes a gel material that is interposed between the patient (P) and the ground return plate. In the present example, ground pad assembly (440) is positioned under the patient (P) and is coupled to power generator (410) via a cable (432) via ports (416, 434). Either or both of ports (416, 434) may include an integral sensor. By way of example only, such a sensor in either or both of ports (416, 434) may be configured to monitor whether excess or inductive energy is radiating from ground pad assembly (440).

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (420), such risks may occur with respect to sensor (426), distal electrode (428), and/or any other electrical components in instrument (420). Other components of instrument (420) that may present the above-described risks will be apparent to those skilled in the art in view of the teachings herein. Such risks may be greater in versions instrument (420) that are dedicated to providing monopolar RF energy than in the context of bipolar RF instruments such as instrument (350) because a dedicated monopolar RF instrument may lack a ground return path that might otherwise prevent or mitigate the above risks.

D. Example of Articulation Section in Shaft Assembly

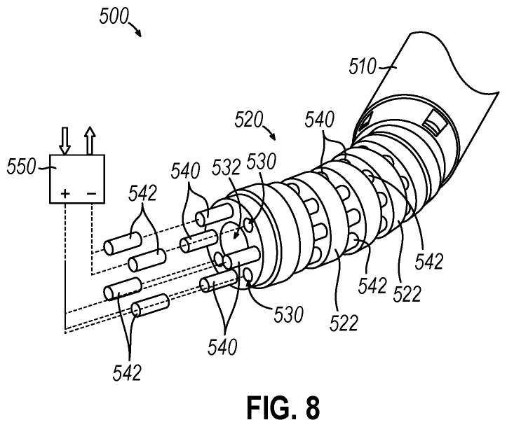

FIG. 8 illustrates a portion of an instrument (500) that includes a shaft (510) with an articulation section (520). In addition to the following teachings, instrument (500) may be constructed and operable in accordance with at least some of the teachings of U.S. Pub. No. 2017/0202591, the disclosure of which is incorporated by reference herein, in its entirety; and/or various other references cited herein. In the present example, an end effector (550) is positioned at the distal end of articulation section (520). Articulation section (520) includes a plurality of segments (522) and is operable to laterally deflect end effector (550) away from and toward the central longitudinal axis of shaft (510). A plurality of wires (540) extend through shaft (510) and along articulation section (520) to reach end effector (550) and thereby deliver electrical power to end effector (550). By way of example only, end effector (550) may be operable to deliver monopolar and/or bipolar RF energy to tissue as described herein. A plurality of push-pull cables (542) also extend through articulation section (520). Push-pull cables (542) may be coupled with an actuator (e.g., similar to articulation control (218)) to drive articulation of articulation section (520). Segments (522) are configured to maintain separation between, and provide structural support to, wires (540) and push-pull cables (542) along the length of articulation section (520). Articulation section (520) of this example also defines a central passageway (532). By way of example only, central passageway (532) may accommodate an acoustic waveguide (e.g., in variations where end effector (550) further includes an ultrasonic blade), may provide a path for fluid communication, or may serve any other suitable purpose. Alternatively, central passageway (532) may be omitted.

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (500), such risks may occur with respect to wires (540) and/or push-pull cables (542). In addition, instrument (500) may include one or more sensors in shaft assembly (510) and/or end effector (550); and may also include one or more electrodes and/or other electrical features in end effector (550). Other components of instrument (500) that may present the above-described risks will be apparent to those skilled in the art in view of the teachings herein.

E. Example of Wiring to End Effector

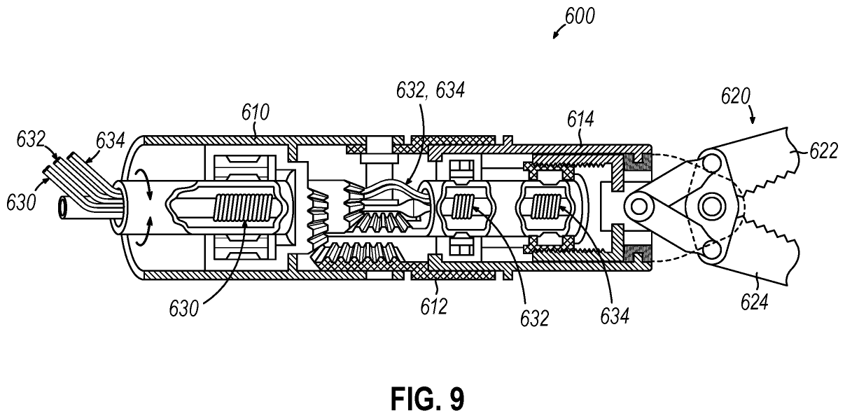

FIG. 9 illustrates a portion of an instrument (600) that includes a shaft (610) with n first articulating segment (612) and a second articulating segment (614). In addition to the following teachings, instrument (600) may be constructed and operable in accordance with at least some of the teachings of U.S. Pub. No. 2017/0202605, entitled “Modular Battery Powered Handheld Surgical Instrument and Methods Therefor,” published Jul. 20, 2017, the disclosure of which is incorporated by reference herein, in its entirety; and/or various other references cited herein. In the present example, end effector (620) is positioned at the distal end of second articulating segment (614). End effector (620) of this example includes a pair of jaws (622, 624) that are operable to pivot toward and away from each other to grasp tissue. In some versions, one or both of jaws (622, 624) includes one or more electrodes that is/are operable to apply RF energy to tissue as described herein. In addition, or in the alternative, end effector (620) may include an ultrasonic blade and/or various other features. Segments (612, 614) may be operable to pivot relative to shaft (610) and relative to each other to thereby deflect end effector (620) laterally away from or toward the central longitudinal axis of shaft (610).

Instrument (900) of this example further includes a first wire set (630) spanning through shaft (610), a second wire set (632) spanning through shaft (610) and both segments (612, 614), and a third wire set (634) spanning further through shaft (610) and both segments (612, 614). Wire sets (630, 632, 634) may be operable to control movement of segments (612, 614) relative to shaft (610). For instance, power may be communicated along one or more of wire sets (630, 632, 634) to selectively engage or disengage corresponding clutching mechanisms, to thereby allow lateral deflection of one or both of segments (612, 614) relative to shaft (610); and or rotation of one or both of segments (612, 614) relative to shaft (610). Alternatively, power may be communicated along one or more of wire sets (630, 632, 634) to drive corresponding solenoids, motors, or other features to actively drive lateral deflection of one or both of segments (612, 614) relative to shaft (610); and or rotation of one or both of segments (612, 614) relative to shaft (610). In versions where end effector (620) is operable to apply RF energy to tissue, one or more additional wires may extend along shaft (610) and segments (612, 614), in addition to wire sets (630, 632, 634).

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (600), such risks may occur with respect to wire sets (630, 632, 634), the electrical components that wire sets (630, 632, 634) are coupled with, and/or other features that drive lateral deflection of one or both of segments (612, 614) relative to shaft (610). In addition, instrument (600) may include one or more sensors in shaft assembly (610) and/or end effector (620); and may also include one or more electrodes and/or other electrical features in end effector (620). Other components of instrument (600) that may present the above-described risks will be apparent to those skilled in the art in view of the teachings herein.

F. Example of Sensors in Shaft Assembly

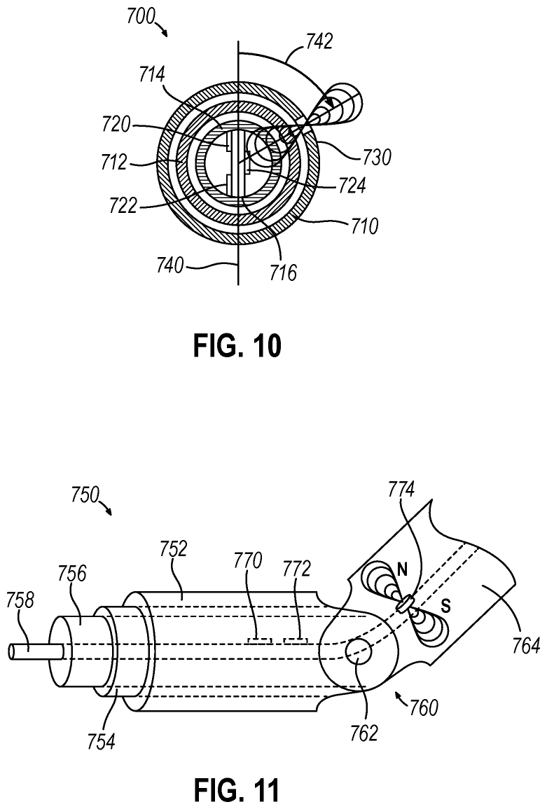

FIG. 10 shows an example of another shaft assembly (700) that may be incorporated into any of the various instruments (40, 50, 60, 100, 190, 200, 300, 350, 400, 500, 600) described herein. In addition to the following teachings, shaft assembly (700) may be constructed and operable in accordance with at least some of the teachings of U.S. Pub. No. 2017/0202608, the disclosure of which is incorporated by reference herein, in its entirety; and/or various other references cited herein. Shaft assembly (700) of this example includes an outer shaft (710), a first inner shaft (712), and a second inner shaft (714). A support member (716) spans diametrically across the interior of second inner shaft (714). By way of example only, support member (716) may comprise a circuit board, a flex-circuit, and/or various other electrical components. A plurality of sensors (720, 722, 724) are positioned on support member (716) in the present example. A magnet (730) is embedded in outer shaft (710) which is operable to rotate about inner shafts (712, 714).

In some versions, rotation of outer shaft (710) about inner shafts (712, 714) drives rotation of an end effector (not shown), located at the distal end of shaft assembly (700), about a longitudinal axis of shaft assembly (700). In some other versions, rotation of outer shaft (710) about inner shafts (712, 714) drives lateral deflection of the end effector away from or toward the longitudinal axis of shaft assembly (700). Alternatively, rotation of outer shaft (710) about inner shafts (712, 714) may provide any other results. In any case, sensors (720, 722, 724) may be configured to track the position of magnet (730) and thereby determine a rotational position (742) of outer shaft (710) relative to a fixed axis (740). Thus, sensors (720, 722, 724) may collectively serve as a position sensor like position sensor (112) of instrument (100).

FIG. 11 shows an example of another shaft assembly (750) that may be incorporated into any of the various instruments (40, 50, 60, 100, 190, 200, 300, 350, 400, 500, 600) described herein. In addition to the following teachings, shaft assembly (750) may be constructed and operable in accordance with at least some of the teachings of U.S. Pub. No. 2017/0202608, the disclosure of which is incorporated by reference herein, in its entirety; and/or various other references cited herein. Shaft assembly (750) of this example includes a plurality of coaxially positioned proximal shaft segments (752, 754, 756) and a distal shaft segment (764). Distal shaft segment (764) is pivotably coupled with proximal shaft segment (752) via a pin (762) to form an articulation joint (760). An end effector (not shown) may be positioned distal to distal shaft segment (764), such that articulation joint (760) may be utilized to deflect the end effector laterally away from or toward a central longitudinal axis defined by proximal shaft segments (752, 754, 756). A flex circuit (758) spans along shaft segments (752, 754, 756, 764) and is operable to flex as shaft assembly (750) bends at articulation joint (760).

A pair of sensors (770, 772) are positioned along flex circuit (758) within the region that is proximal to articulation joint (760); while a magnet (774) is positioned on flex circuit (758) (or elsewhere within distal shaft segment (764)) in the region that is distal to articulation joint (760). Magnet (774) thus moves with distal shaft segment (764) as distal shaft segment (764) pivots relative to proximal shaft segments (752, 754, 756) at articulation joint (760); while sensors (770, 772) remain stationary during such pivoting. Sensors (770, 772) are configured to track the position of magnet (774) and thereby determine a pivotal position of distal shaft segment (764) relative to proximal shaft segments (752, 754, 756). In other words, sensors (770, 772) and magnet (774) cooperate to enable determination of the articulation bend angle formed by shaft assembly (750). Thus, sensors (770, 772) may collectively serve as a position sensor like position sensor (112) of instrument (100).

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instruments (700, 750), such risks may occur with respect to sensors (720, 722, 724, 770, 772), the electrical components that sensors (720, 722, 724, 770, 772) are coupled with, and/or other features within the shaft assemblies of instruments (700, 750). Other components of instruments (700, 750) that may present the above-described risks will be apparent to those skilled in the art in view of the teachings herein.

G. Example of Drive Controls in Body and Shaft Assembly of Instrument

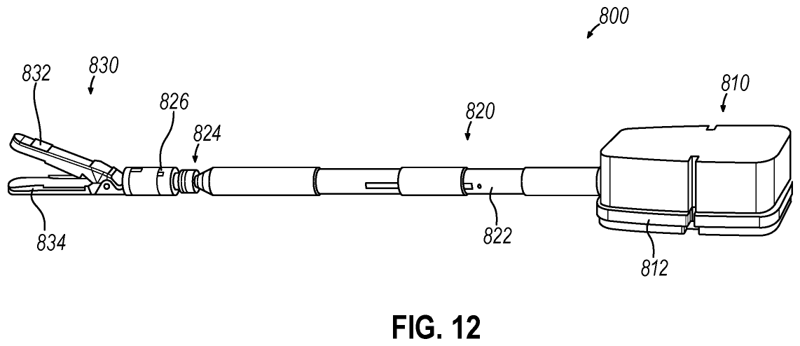

FIGS. 12-14 show an example of an instrument (800) that may be incorporated into a robotic surgical system, such as the robotic surgical systems (10, 150) described herein. In addition to the following teachings, instrument (800) may be constructed and operable in accordance with at least some of the teachings of U.S. Pat. No. 9,125,662, the disclosure of which is incorporated by reference herein, in its entirety; and/or various other references cited herein. Instrument (800) of this example includes a body (810), a shaft assembly (820), and an end effector (830). Body (810) includes a base (812) that is configured to couple with a complementary component of a robotic arm (e.g., one of robotic arms (160, 170, 180)). Shaft assembly (820) includes a rigid proximal portion (822), an articulation section (824), and a distal portion (826). End effector (830) is secured to distal portion (826). Articulation section (824) is operable to deflect distal portion (826) and end effector (830) laterally away from and toward the central longitudinal axis defined by proximal portion (822). End effector (830) of this example includes a pair of jaws (832, 834). By way of example only, end effector (830) may be configured and operable like any of the various end effectors (46, 56, 66, 102, 230, 320, 350, 620) described herein.

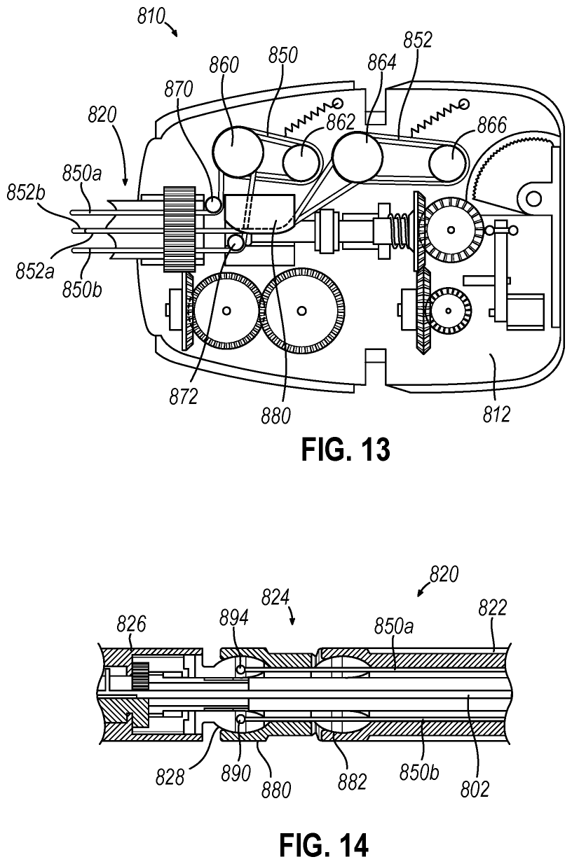

As shown in FIGS. 13-14, a plurality of drive cables (850, 852) extend from body (810) to articulation section (824) to drive articulation of articulation section (824). Cable (850) is wrapped around a drive pulley (862) and a tensioner (860). Cable (850) further extends around a pair of guides (870, 872), such that cable (850) extends along shaft assembly (820) in two segments (850a, 850b). Cable (852) is wrapped around a drive pulley (866) and a tensioner (864). Cable (852) further extends around a guide (880), such that cable (852) extends along shaft assembly (820) in two segments (852a, 852b). In the present example, each drive pulley (862, 866) is configured to couple with a corresponding drive member (e.g., drive spindle, etc.) of the component of the robotic arm to which base (812) is secured. When drive pulley (862) is rotated, one segment (850a) of cable (850) will translate in a first longitudinal direction along shaft assembly (820); while the other segment (850b) will simultaneously translate in a second (opposite) direction along shaft assembly (820). Similarly, when drive pulley (866) is rotated, one segment (852a) of cable (852) will translate in a first longitudinal direction along shaft assembly (820); while the other segment (852b) will simultaneously translate in a second (opposite) direction along shaft assembly (820).

As shown in FIG. 14, articulation section (824) of the present example includes an intermediate shaft segment (880) that is longitudinally interposed between proximal portion (822) and distal portion (826). A ball feature (828) at the proximal end of distal portion (826) is seated in a socket at the distal end of intermediate shaft segment (880), such that distal portion (826) is operable to pivot relative to intermediate shaft segment (880) along one or more planes. Segments (850a, 850b) of drive cable (850) terminate in corresponding ball-ends (894, 890), which are secured to ball feature (828) of distal portion (822). Drive cable (850) is thus operable to drive pivotal movement of distal portion (826) relative to intermediate shaft segment (880) based on the direction in which drive pulley (862) rotates. A ball feature (882) at the proximal end of intermediate portion (880) is seated in a socket at the distal end of proximal portion (822), such that intermediate portion (880) is operable to pivot relative to proximal portion (822) along one or more planes. In some versions, this pivotal movement of intermediate portion (880) relative to proximal portion (822) is driven by cable (852). As also shown in FIG. 14, an electrical cable (802) passes through articulation section (824). Electrical cable (802) provides a path for electrical communication to end effector (830), thereby allowing for delivery of electrical power (e.g., RF energy) to one or more electrodes in end effector (830), providing a path for electrical signals from one or more sensors in end effector (830) to be communicated back to body (810), and/or other forms of electrical communication.

As noted above, instruments (150, 200) may include electrical features and/or electrically conductive mechanical features that may provide a risk of power or signals undesirably crossing from one electrical feature to another electrical feature and/or from one electrical feature to an electrically conductive mechanical feature. In addition, instruments (150, 200) may include electrical features and/or electrically conductive mechanical features that may provide a risk of generating electrical potentials between proximate components or creating capacitive couplings between electrical features and/or between an electrical feature and an electrically conductive mechanical feature. In the context of instrument (800), such risks may occur with respect to drive cables (850, 852), the components that (850, 852) are coupled with, electrical features within shaft assembly (820), and/or other features within instrument (800). Other components of instrument (800) that may present the above-described risks will be apparent to those skilled in the art in view of the teachings herein.

H. Example of Electrical Features at Interface between Modular Components of Instrument

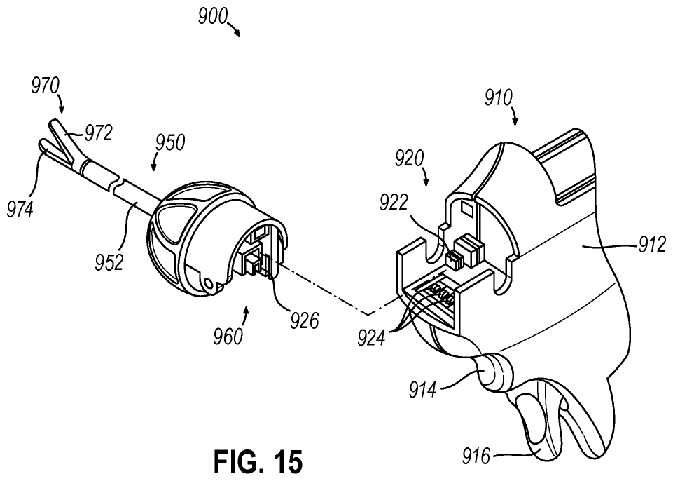

In some instances, it may be desirable to provide a surgical instrument that allows for modular coupling and decoupling of components. For instance, FIG. 15 shows an example of an instrument (900) that includes a handle assembly (910) and a modular shaft assembly (950). While instrument (900) of this example is handheld, similar features and modularity may be readily incorporated into a robotically controlled instrument. Handle assembly (910) of this example includes a body (912), an activation button (914), a pivoting trigger (916), and a shaft interface assembly (920). Shaft interface assembly (920) includes a mechanical drive feature (922) and an array of electrical contacts (924). Electrical contacts (924) may be in electrical communication with a control circuit, power source, and/or various other electrical features within handle assembly (910) as will be apparent to those skilled in the art in view of the teachings herein.

Shaft assembly (950) includes a shaft section (952) and an end effector (970), which includes a pair of jaws (972, 874). Shaft section (952) and end effector (970) may be configured and operable in accordance with any of the various shaft assemblies and end effectors described herein. Shaft assembly (950) of this example further includes a handle interface assembly (960). Handle interface assembly (960) includes a mechanical drive feature (962) and a plurality of electrical contacts (not shown). These electrical contacts of handle interface assembly (960) may be in electrical communication with one or more electrodes, sensors, and/or other electrical components within shaft section (952) and/or end effector (970) as will be apparent to those skilled in the art in view of the teachings herein.

When shaft assembly (950) is coupled with handle assembly (910), mechanical drive feature (922) of handle assembly (910) mechanically couples with mechanical drive feature (962) of shaft assembly (950), such that mechanical drive features (922, 962) may cooperate to communicate motion from a motive power source in handle assembly (910) (e.g., pivoting trigger (916), a motor, etc.) to one or more components within shaft section (952) and, in some versions, end effector (970). In some versions, mechanical drive features (922, 962) cooperate to communicate rotary motion from a motive power source in handle assembly (910) (e.g., pivoting trigger (916), a motor, etc.) to one or more components within shaft section (952) and, in some versions, end effector (970). In addition, or in the alternative, mechanical drive features (922, 962) may cooperate to communicate linear translational motion from a motive power source in handle assembly (910) (e.g., pivoting trigger (916), a motor, etc.) to one or more components within shaft section (952) and, in some versions, end effector (970).

When shaft assembly (950) is coupled with handle assembly (910), electrical contacts (924) of shaft interface assembly (920) also couple with complementary electrical contacts of handle interface assembly (960), such that these contacts establish continuity with each other and thereby enable the communication of electrical power, signals, etc. between handle assembly (910) and shaft assembly (950). In addition to or in lieu of having contacts (924), electrical continuity may be provided between handle assembly (910) and shaft assembly (950) via one or more electrical couplings at mechanical drive features (922, 962). Such electrical couplings may include slip couplings and/or various other kinds of couplings as will be apparent to those skilled in the art in view of the teachings herein.

In some scenarios where electrical power or electrical signals are communicated across mating contacts that provide electrical continuity between two components of an instrument (e.g., contacts (924) of shaft interface assembly (920) and complementary electrical contacts of handle interface assembly (960)), there may be a risk of short circuits forming between such contacts. This may be a particular risk when contacts that are supposed to be electrically isolated from each other are located in close proximity with each other, and the area in which these contacts are located may be exposed to fluids during use of the instrument. Such fluid may create electrical bridges between contacts and/or bleed signals that are being communicated between contacts that are supposed to be coupled with each other. It may therefore be desirable to provide features to prevent or otherwise address such occurrences at contacts of an instrument like instrument (900).

In some scenarios where electrical power or electrical signals are communicated across mechanical couplings between different components of an instrument (e.g., via slip couplings, etc.), such couplings might provide variable electrical resistance in a shaft assembly or other assembly of the instrument. For instance, motion at mechanical drive features (922, 962) may provide variable electrical resistance at an electrical slip coupling between mechanical drive features (922, 962); and this variable electrical resistance may impact the communication of electrical power or electrical signals across the slip coupling. This may in turn result in signal loss or power reductions. It may therefore be desirable to provide features to prevent or otherwise address such occurrences at electrical couplings that are found at mechanical couplings between two moving parts of an instrument like instrument (900).

### IV. EXAMPLES OF ELECTROSURGICAL SYSTEM POWER MONITORING FEATURES

The following description relates to examples of different features that may be incorporated into any of the various RF electrosurgical instruments (40, 50, 420) described above. While these examples are provided separate from each other, the features described in any of the following examples may be combined with the features described in other examples described herein. Thus, the below-described features may be combined in various permutations as will be apparent to those skilled in the art in view of the teachings herein. Similarly, various ways in which the below-described features may be incorporated into any of the various instruments (40, 50, 420) described above will be apparent to those skilled in the art in view of the teachings herein. It should be understood that the below-described features may be incorporated into robotically controlled surgical instruments and/or handheld surgical instruments, including but not limited to such instruments that are powered via on-board battery and/or powered via wire to an external power source. This includes, but is not limited to, the various kinds of robotically controlled instruments described above, the various kinds of handheld instruments described above, the various kinds of battery-powered instruments described above, and the various kinds of instruments described above that are powered via wire to an external power source.

As noted above, some aspects of the present disclosure are presented for a surgical instrument with improved device capabilities for reducing undesired operational side effects. Examples of such devices and related concepts are disclosed in U.S. Pat. Pub. No. 2019/0201077, entitled “Interruption of Energy Due to Inadvertent Capacitive Coupling,” published Jul. 4, 2019, the disclosure of which is incorporated by reference herein. In particular, the surgical instrument may include means for limiting capacitive coupling to improve monopolar RF isolation for use independently or in cooperation with another advanced energy modality. Capacitive coupling occurs generally when there is a transfer of energy between nodes, induced by an electric field. During surgery, capacitive coupling may occur when two or more electrical surgical instruments are being used in or around a patient. Capacitive coupling may also occur within a single instrument or single instrument system. For instance, capacitive coupling may occur between electrically conductive components that are in close proximity with each other in the same instrument, including such components as described above with reference to FIGS. 1-15. While in some cases capacitive coupling may be desirable, as additional devices may be powered inductively by capacitive coupling, having capacitive coupling occur accidentally during surgery or around a patient generally can have extremely deleterious consequences.

Parasitic or accidental capacitive coupling may occur in unknown or unpredictable locations, causing energy to be applied to unintended areas. When the patient is under anesthesia and unable to provide any response, parasitic capacitive coupling may cause undesired thermal damage to a patient before the operator realizes that any thermal damage is occurring. In addition, or in the alternative, parasitic capacitive coupling may result in undesirable electrical power losses. Such undesirable electrical power losses due to parasitic capacitive coupling may result in undesirably low delivery of electrical energy (e.g., monopolar RF energy) to tissue in the patient, which may produce an undesirable surgical result. In addition, or in the alternative, undesirable electrical power losses due to parasitic capacitive coupling may result in compromised feedback signals from sensors or other electrical components, where such adversely affected electrical signals result in unreliable feedback data. It is therefore desirable to prevent or at least limit parasitic or accidental capacitive coupling in surgical instruments and during surgery generally.

In some versions of the instruments described above, the electrosurgical system includes a surgical instrument and console, such as console (20) (see, FIG. 1). The console may include data processors, memory, and other computer equipment, along with one or more generators. Each generator may be configured to modulate the transmission of energy from the generator to the particular surgical instrument that the generator is powering if capacitive coupling has been detected along any of the components coupled with that surgical instrument. One or more safety fuses, sensors, controls, and/or algorithms may be in place to automatically trigger a modulation of the energy delivered by the generator in these scenarios. Alerts, including audio signals, vibrations, and visual messages may issue to inform the surgery team that the energy has been modulated, or is being modulated, due to the detection of capacitive coupling.

In some aspects, the system includes means for detecting that a capacitive coupling event has occurred. For example, an algorithm that includes inputs from one or more sensors for monitoring events around the system may apply situational awareness and other programmatic means to conclude that capacitive coupling is occurring somewhere within the system and react accordingly. A system having situational awareness means that the system may be configured to anticipate scenarios that may arise based on present environmental and system data and determining that the present conditions follow a pattern that gives rise to predictable next steps. As an example, the system may apply situational awareness in the context of handling capacitive coupling events by recalling instances in similarly situated surgeries where various sensor data is detected. The sensor data may indicate an increase in current at two particular locations along a closed loop electrosurgical system, that based on previous data of similarly situated surgeries, indicates a high likelihood that a capacitive coupling event is imminent.

In some aspects, the surgical instruments may be modified in structure to limit the occurrence of capacitive coupling, or in other cases reduce the collateral damage caused by capacitive coupling. For example, additional insulation placed strategically in or around the surgical instrument may help limit the incidence of capacitive coupling. In other cases, the end effector of the surgical instrument may include modified structures that reduce the incidence of current displacement, such as rounding the tips of the end effector or specifically shaping the blade of the end effector to behave more like a monopolar blade while still acting as a bipolar device.

In some aspects, the system may include passive means for mitigating or limiting the effects of the capacitive coupling. For example, the system may include leads that can shunt the energy to a neutral node through conductive passive components. In general, any and all of these aspects may be combined or included in a single system to address the challenges posed by multiple electrical components liable to cause capacitive coupling during patient surgery.

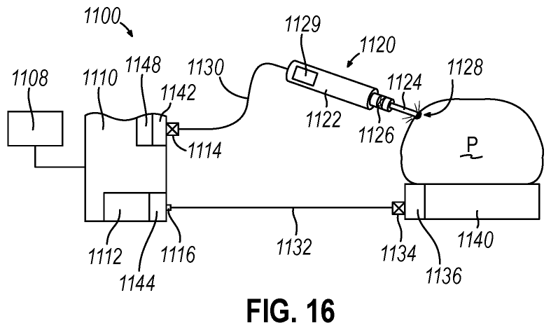

In scenarios where there are multiple electrical sources near patient (P) and/or multiple electrically conductive components within an instrument in close proximity to electrical power-carrying components in the same instrument, parasitic capacitive coupling may present risks to a during surgery. Because patient (P) is not expected to express any reaction during surgery, if unknown or unpredicted capacitive coupling occurs, patient (P) may experience burns in unintended places as a result. In general, energy anomalies like capacitive coupling should be minimized or otherwise corrected in order to improve patient safety and/or otherwise provide desired surgical results. To monitor the occurrence of capacitive coupling or other types of energy anomalies, multiple smart sensors may be integrated into an electrosurgical system as indicators to determine whether excess or inductive energy is radiating outside the one or more of the electrical sources. An example of a system (1100) that incorporates such smart sensors is shown in FIG. 16. System (1100) of FIG. 16 is substantially similar to system (400) of FIG. 7, described above, but with variations described below.

System (1100) of FIG. 16 is operable to detect capacitive couplings that inadvertently occur within or between components of system (1100), in accordance with at least one aspect of the present disclosure. System (1100) of this example includes a power generator (1110), a delivery instrument (1120), and a ground pad assembly (1140). Instrument (1120) of system (1100) may include means for applying RF or ultrasonic energy to a distal electrode (1128), and in some cases may include a blade and/or a pair of jaws to grasp or clamp onto tissue. In addition to the following teachings, instrument (1120) may be constructed and operable in accordance with at least some of the teachings of U.S. Pub. No. 2019/0201077, the disclosure of which is incorporated by reference herein, in its entirety; and/or various other references cited herein.

Power generator (1110) may be operable to deliver monopolar RF energy to instrument (1120) via a cable (1130), which is coupled with power generator (1110) via a port (1114). The energy powered by the generator (1110) may touch the patient (P) through distal electrode (1128) of instrument (1120). In the present example, port (1114) includes an integral sensor (1142) and a tuner (1148). By way of example only, sensor (1142) in port (1114) may be configured to monitor whether excess or inductive energy is radiating from power generator (1110) and/or whether parasitic losses are occurring in energy being delivered by power generator (1110). Tuner (1148) may be configured to modulate the delivery of energy by power generator (1110) via port (1114), based at least in part on feedback from sensor (1142). Examples of how such modulation may be carried out will be described in greater detail below.

Instrument (1120) includes a body (1122), a shaft (1124), a sensor (1126), and a distal electrode (1128) that is configured to contact a patient (P) and thereby apply monopolar RF energy to the patient (P). By way of example only, sensor (1126) may be configured to monitor whether excess or inductive energy is radiating from instrument (1120) and/or whether parasitic losses are occurring in signals from instrument (1120). Based on feedback signals from sensor (1126), a control module in power generator (1110) may passively throttle or otherwise adjust the ground return from ground pad assembly (1140). In addition, or in the alternative, the ground return from ground pad assembly (1140) may me throttled or otherwise adjusted based at least in part on feedback from sensor (1142) and/or other sources.

Ground pad assembly (1140) is configured to provide an electrical ground to the patient (P) when surgical instrument (1120) touches patient (P) and applies electrosurgical energy to the patient (P). In this role, ground pad assembly (1140) may further divert excess energy (e.g., undesirable excess electrosurgical energy) that is undesirably delivered to the patient (P). In some versions, ground pad assembly (1140) comprises one or more resistive continuity ground pads that provide direct contact between the skin of the patient (P) and one or more metallic components of the ground pad. In some other versions, ground pad assembly (1140) comprises a capacitive coupling ground pad that includes a gel material that is interposed between the patient (P) and the ground return plate. By way of example only, ground pad assembly (1140) may be configured and operable similar to a Smart MEGADYNE™ MEGA SOFT™ pad by Ethicon US, LLC. In the present example, ground pad assembly (1140) is positioned under the patient (P) and is coupled to a neutral electrode (1112) of power generator (1110) via a cable (1132). Cable (1132) is coupled via ports (1116, 1134). Either or both of ports (1116, 1134) may include an integral sensor (1144, 1146). By way of example only, such a sensor (1144, 1146) in either or both of ports (1116, 1134) may be configured to monitor whether excess or inductive energy is radiating from ground pad assembly (1140). Based on feedback signals from one or both of sensors (1144, 1146), a control module in power generator (1110) may passively throttle or otherwise adjust the ground return from ground pad assembly (1140).

As shown in FIG. 16, sensors (1126, 1142, 1144, 1146) of the present example are placed at locations where energy may inductively radiate. One or more of sensors (1126, 1142, 1144, 1146) may be configured to detect capacitance; and if placed at strategic locations within system (1100), a reading of capacitance may imply that capacitive leakage is occurring near the sensor (1126, 1142, 1144, 1146). With knowledge of other sensors nearby or throughout the system not indicating a reading of capacitance, one may conclude that capacitive leakage is occurring in close proximity to whichever sensor (1126, 1142, 1144, 1146) is providing a positive indication. Other sensors may be used, such as capacitive leakage monitors or detectors. These sensors may be configured to provide an alert, such as lighting up or delivering a noise or transmitting a signal ultimately to a display monitor. In addition, generator (1110) may be configured to automatically modulate the energy being delivered via port (1114) to stop any further capacitive coupling from occurring.

In some aspects, generator (1110) may be configured to employ situational awareness that can help anticipate when capacitive coupling may occur during surgery. Generator (1110) may utilize a capacitive coupling algorithm to monitor the incidence of energy flowing through system (1100), and based on previous data about the state of energy in the system for a similar situated procedure, may conclude there is a likelihood that capacitive coupling may occur if no additional action is taken. For example, during a surgery involving prescribed methods for how to operate instrument (1120) and how much power should be employed during particular steps in the surgery, generator (1110) may draw from previous surgeries of the same and note that capacitive coupling has a stronger likelihood to occur after a particular step in the surgery. While monitoring the steps in the surgery, when the same or very similar energy profiles occur during or just before the expected step that tends to induce capacitive coupling, generator (1110) may deliver an alert that indicates this is likely to cause capacitive coupling. The operator may be given the option to reduce peak voltage in surgical instrument (1120), interrupt the power generation by generator (1110), or otherwise modulate the delivery of power from generator (1110) to instrument (1120). This may lead to eliminating the possibility of capacitive coupling before it has a chance to occur, or at least may limit any unintended effects caused by a momentary occurrence of capacitive coupling.

In some aspects, surgical instrument (1120) may include structural means for reducing or preventing capacitive coupling. For example, insulation in shaft (1124) of surgical instrument (1120) may reduce the incidence of inductance. In other cases, wire (1130) connecting generator (1110) to instrument (1120) or components on or within body (1122) may be shielded and coupled with a ground source, such as back through cable (1130) or by coupling with return path cable (1132) (not shown). Sensor (1142) may be further configured to sense the current returning to generator (1110) or other ground source through cable (1130) in addition to sensing the power output to electrode (1128). As another example, interrupting plastic elements within shaft (1124) may be intermittently present to prevent capacitive coupling from transmitting long distance within the shaft. Other insulator-type elements may be used to achieve similar effects.

As described above, some existing instruments may be configured to interrupt the power generation by the generator upon detecting capacitive coupling at one or more sensors. While such power interruptions may be effective in preventing the occurrence of undesirable results that might otherwise occur due to inadvertent capacitive coupling, such power interruptions may be disfavored by an operator of instrument (1120), particularly when the power interruption occurs suddenly during the middle of a surgical procedure. Power interruptions during a surgical procedure may frustrate the operator and increase the duration of surgery. It may therefore be more desirable to modulate the power delivered from a generator (1110) to an instrument (1120), without interrupting the power, to prevent the occurrence of undesirable results that might otherwise occur due to inadvertent capacitive coupling. Such power modulation may be provided on an ad hoc basis in response to real time feedback from sensors as described herein. While the exemplary methods will be described below with continued reference to system (1100), it should be understood that the methods described herein may be incorporated into other electrosurgical systems which may include sensors for monitoring capacitive leakage, including systems that provide modes of power delivery that are not necessarily limited to monopolar RF power delivery.

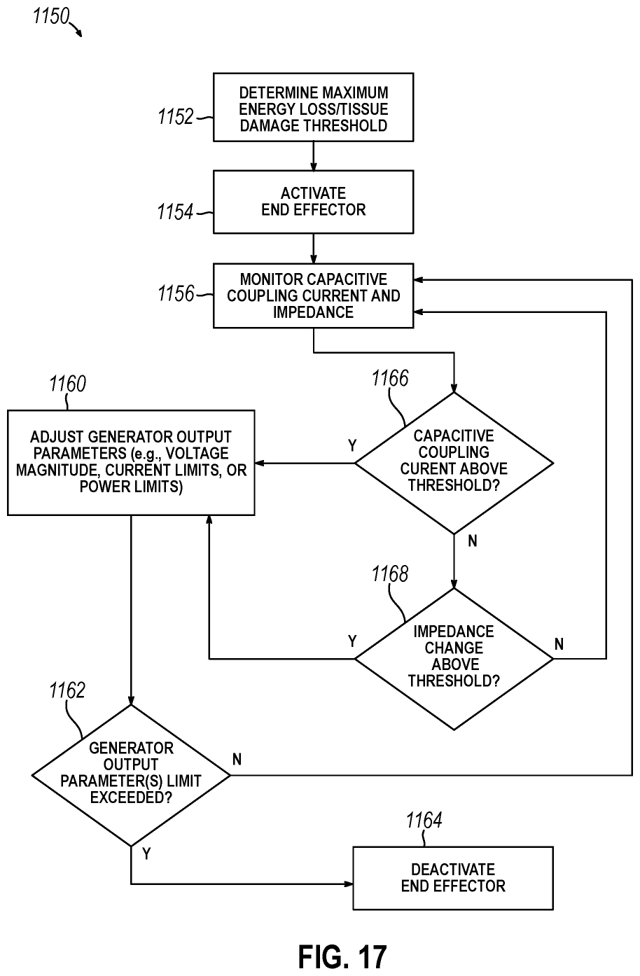

FIG. 17 depicts a flowchart of an exemplary method of monitoring the energy loss of a surgical instrument that is operable to apply RF energy to tissue, such as any one of instruments (40, 50, 420, 1120) described herein. By employing the exemplary methods, such as within system (1100), one or more of sensors (1126, 1142, 1144, 1146) (see FIG. 16) are configured to monitor the capacitive coupling currents and instrument impedance and to provide feedback to generator (1110) (or, alternatively, to a data processor of console (20) that is controlling generator (1110)). Generator (1110), or a local or cloud-based processing device coupled with generator (1110) for example, is then able to determine whether generator (1110) should increase or decrease the voltage delivered to electrode (1128) of instrument (1120). If capacitive coupling current is at or above a pre-determined threshold current, generator (1110) may be directed to turn down the voltage to therefore decrease the capacitive redirection to a level that is below the injury threshold but still allows instrument (1120) and the operator to operate. Otherwise, if capacitive coupling energy is below the pre-determined threshold, generator (1110) may be directed to turn up the voltage to provide more power to instrument (1120) while still monitoring the threshold for capacitive coupling. Thus, by monitoring the level of capacitive coupling (e.g., too much leakage) rather than solely monitoring for the presence or absence of capacitive coupling, system (1100) is able to track the aberrant energy redirection as generator (1110) adjusts the voltage from potentially a high voltage power usage (e.g., 7,000 volts) to a significantly lower voltage (e.g., 1,000 volts) while still maintaining the same power level by simultaneously adjusting the output current. As these adjustments are made, generator (1110), sensors (1126, 1142, 1144, 1146) or other monitoring devices monitor the aberrant capacitive coupling current to ensure that the capacitive coupling current moves below a tissue damaging threshold level, at which point the adjustment allows instrument (1120) to continue to be used in the operation. In other words, capacitive coupling may be suitably addressed without requiring the surgical procedure to stop due to sudden interruption of power from generator (1110). In some cases, however, where ad hoc power modulation will not suffice to address capacitive coupling, it may ultimately be desirable to interrupt power from generator (1110) as a last resort.

If output energy from instrument (1120) is capacitively coupled to tissue of patient (P), a lower impedance load may be seen by generator (1110) relative to the impedance load provided by the tissue alone without the capacitive coupling. Monitoring abrupt changes in impedance could signal harmful arcing or breakdown. Thus, generator (1110) may be monitored for arcing, data of which may be used cooperatively with local electronics in instrument (1120) to better evaluate what percentage of the output power is being delivered to electrode (1128) versus to the capacitive coupling. This may allow the monitoring systems to provide feedback for generator (1110) output adjustments actively in real-time during an operation, thereby allowing generator (1110) to adjust the voltage or other electrical parameter(s) as necessary. In some versions, a shielding (1129) is included in instrument (1120) to collect capacitive coupling current to provide to sensors (1126, 1142, 1144, 1146) for measurements and monitoring. System (1100) may include controller (1108) (e.g., a hub or data center) having processing means for coupling with generator (1110); or the processing means may be included within generator (1110). The electrosurgery parameters may therefore be measured by sensors (1126, 1142, 1144, 1146) and compared, by the processor, with an estimate of what a normal application of energy or a normal tissue impedance would be for the operative situation. If either parameter is out of a pre-determined range, then generator (1110) may be made aware that there is the possibility of capacitive coupling or a breakdown of the insulation system on the instrument.

As an alternative, tuner (1148) may be coupled with output port (1114) to adjust the capacitive and/or inductive load automatically to therefore adjust for higher or lower capacitance components of instrument (1120), such as a metallic shield (1129) that is in, on, or around at least a portion of instrument (1120). Components could be measured upon connection of instrument (1120) and then adjustments made to compensate. In addition or in the alternative, as exceedingly high voltages are sensed by one or more sensors (1126, 1142, 1144, 1146), system (1100) may add or subtract some capacitance and/or inductance to reduce the energy output at port (1114).

As depicted in FIG. 17, an example of a method (1150) as described above begins with a step (block 1152) where one or more sensors (1126, 1142, 1144, 1146) determine the maximum threshold or range of energy loss allowable and/or the maximum threshold or range of impedance change allowable during the operation. These thresholds or ranges may be determined by system (1100), such as by controller (1108) or generator (1110), based upon known parameters of the surgical operation at hand, based on known parameters of instrument (1120), based on prior operation data collected from similar surgical operations or with similar instruments, and/or based on other factors. In some versions, tuner (1142) automatically executes a calibration algorithm upon coupling of instrument (1120) with generator (1110) to detect the load parameters of the coupled instrument (1120), and thereby determines appropriate the maximum threshold or range of energy loss allowable and/or the maximum threshold or range of impedance change allowable during the operation, based on the detected load parameters of the coupled instrument (1120). Such ad hoc determinations may further allow for power delivery adjustments to be made before the power is even initially delivered, to compensate for the detected load parameters of the coupled instrument (1120). By way of example only, such initial ad hoc power delivery adjustments may include adding or subtracting capacitance and/or inductance to the output that will be delivered to the coupled instrument (1120), to thereby minimize the risks of capacitive couplings occurring during use of the coupled instrument (1120) during the surgical procedure. Regardless of whether initial ad hoc power delivery adjustments are made based on detected characteristics of the coupled instrument (1120), the maximum energy loss threshold or range that is determined (block 1152), and the maximum threshold or range of impedance change that is determined (block 1152), may each be configured such that system (1100) directs generator (1110) to adjust the power output of generator (1110) as required to ensure that instrument (1120) operates effectively and patient (P) injury is avoided.

Once the thresholds or ranges are determined, at a next step (block 1154), the operator activates end effector (e.g., electrode (1128)) of instrument (1120) to begin the operation on patient (P). As described above, at a subsequent step (block 1156), one or more of sensors (1126, 1142, 1144, 1146) monitor the capacitive coupling current induced along the components of instrument (1120) and/or wire (1130). During this same step (block 1156), the impedance may also be monitored.

Based on the data from one or more sensors (1126, 1142, 1144, 1146), method (1150) further includes a step of determining (block 1166), via controller (1108) or generator (1110), whether the capacitive coupling current meets or exceeds the threshold or range that was previously determined (block 1152). If the capacitive coupling current does not meet or exceed the threshold or range that was previously determined (block 1152), method (1150) further includes a step of determining (block 1168), via controller or generator (1110), whether the impedance change has meets or exceeds the threshold or range that was previously determined (block 1152), where such an impedance change would be indicative of an undesirable capacitive coupling. For instance, an abrupt and substantial reduction in impedance may indicate undesirable arcing between electrode (1128) and tissue, which may be a result of undesirable capacitive coupling. If neither the capacitive coupling current nor the impedance change has met or exceeded the corresponding threshold or range that was previously determined (block 1152), then system (1100) continues activation of the end effector (block 1154) and monitoring capacitive coupling current and/or impedance (block 1156).

If the determination (block 1166) reveals that the capacitive coupling current meets or exceeds the threshold or range that was previously determined (block 1152), then method (1150) proceeds to a step (block 1160) where one or more output parameters (e.g., voltage magnitude, current limit, power limit, etc.) of generator (1110) are adjusted to prevent or otherwise address the occurrence of capacitive coupling. Similarly, if the determination (block 1168) reveals that the impedance change meets or exceeds the threshold or range that was previously determined (block 1152), then method (1150) proceeds to a step (block 1160) where one or more output parameters (e.g., voltage magnitude, current limit, power limit, etc.) of generator (1110) are adjusted to prevent or otherwise address the occurrence of capacitive coupling. Such adjustments may be executed via tuner (1148), as described above. In some scenarios, such adjustments include reducing the output voltage of generator (1110) while still maintaining substantially the same power level (despite the reduction of voltage).

After adjusting the output parameters of generator (1110) (block 1160), system (1100) may determine (block 1162) whether these adjusted output parameters exceed the appropriate limits. If the adjusted output parameters do not exceed the appropriate limits, then system (1100) may continue activation of the end effector (block 1154) and monitoring capacitive coupling current and/or impedance (block 1156). The operator may thus continue the surgical procedure without interruption, with system (1100) providing ad hoc adjustments to power delivery from generator (1110), based on real-time feedback from one or more sensors (1126, 1142, 1144, 1146), to prevent undesirable results that might otherwise occur due to capacitive coupling during operation of instrument (1120).

In the event that systems (1100) determines (block 1162) that the adjusted output parameters exceed the appropriate limits, this may mean that system (1100) is unable to make appropriate adjustments to the energy delivered by generator (1110) to instrument (1120) to avoid undesirable results from capacitive coupling. In such scenarios, as a last resort, method (1150) may provide deactivation of the end effector of instrument (1120) (block 1164). Such deactivation may be provided by ceasing or otherwise interrupting energy delivery from generator (1110) to instrument (1120). In some variations, this deactivation (block 1164) may be provided for a predetermined duration (e.g., one second, five seconds, one minute, five minutes, etc.). After the expiry of this predetermined duration, the method may start back with activation of end effector (1154), allowing the surgical procedure to continue once again in accordance with method (1150). In the event that deactivation (block 1164) is necessary, system (1100) may also provide some kind of alert to the operator to indicate that such deactivation (block 1164) is intentional, to thereby avoid confusion by the operator mistakenly thinking that system (1100) has malfunctioned or that some other power failure has occurred. Such an alert may take the form of a visual alert, an audible alert, a haptic alert, and/or combinations of such forms.

### V. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method for performing an electrosurgical procedure using an instrument system, wherein the instrument system includes (a) a surgical instrument having an electrode configured to operate on a tissue of a patient, (b) a generator for powering the electrode, and (c) one or more sensors configured to measure electrical energy flowing between the generator and the patient, the method comprising: (a) determining an electrical parameter threshold of capacitive coupling for monitoring on a conductive component of the surgical instrument during an operation; (b) activating the electrode of the surgical instrument by applying an output power signal from the generator to the electrode, wherein the output power signal has a first energy output profile; (c) monitoring an induced electrical parameter on the conductive component of the surgical instrument via the one or more sensors, the induced electrical parameter being associated with the determined electrical parameter threshold, wherein the induced electrical parameter includes a parasitic energy loss; and (d) when the induced electrical parameter measured from the conductive component of the surgical instrument meets or exceeds the electrical parameter threshold during the operation, adjusting the output power signal of the generator from the first energy output profile to a second energy output profile, wherein the adjustment is operable to reduce the induced electrical parameter measured from the conductive component of the surgical instrument, wherein the adjustment is further operable to reduce the parasitic energy loss without ceasing delivery of energy to the electrode.

### Example 2

The method of Example 1, wherein the conductive component of the surgical instrument is configured to avoid coming into contact with the patient during the operation, the conductive component being separate from the electrode.

### Example 3

The method of any one or more of Examples 1 through 2, wherein a first sensor of the one or more sensors is configured to measure electrical energy communicated from the generator to the patient, wherein a second sensor of the one or more sensors is configured to measure electrical energy communicated from the patient to the generator, wherein the instrument system is configured to measure an impedance of the patient between the first and second sensors, the method further comprising: (a) determining an impedance change threshold for monitoring during an operation; (b) monitoring for a change in the impedance of the patient between the first and second sensors; and (c) when the change of the impedance of the patient meets or exceeds the impedance change threshold during the operation, adjusting the output power signal of the generator from the first energy output profile to the second energy output profile.

### Example 4

The method of any one or more of Examples 1 through 3, wherein adjusting the output power signal includes adjusting at least one of a voltage magnitude, a current limit, or a power limit.

### Example 5

The method of any one or more of Examples 1 through 4, further comprising: (a) upon adjusting the output power signal from the first energy output profile to the second energy output profile, determining whether the generator has reached a power output adjustment limit and is thereby incapable of adjusting the output power signal from the first energy output profile to the second energy output profile; and (b) if the generator has reached the power adjustment limit, disconnecting the output power signal from the electrode.

### Example 6

The method of any one or more of Examples 1 through 5, wherein the conductive component of the surgical instrument includes a metallic shield.

### Example 7

The method of any one or more of Examples 1 through 6, further comprising: (a) prior to activating the electrode of the surgical instrument, positioning a ground electrode on the patient so as to create a current path in the tissue of the patient between the electrode and the ground electrode, wherein the ground electrode includes an electrical lead coupled with an electrical ground node.

### Example 8

The method of any one or more of Examples 1 through 7, wherein the generator is configured to apply monopolar RF energy to the patient.

### Example 9

The method of any one or more of Examples 1 through 8, wherein the surgical instrument is a handheld instrument.

### Example 10

The method of any one or more of Examples 1 through 9, wherein the surgical instrument is a component of a robotic electrosurgical system.

### Example 11

The method of any one or more of Examples 1 through 10, wherein the instrument system further includes a tuner coupled with the generator, wherein the tuner is selectively operable to adjust the output power signal of the generator, wherein adjusting the output power signal of the generator from the first energy output profile to a second energy output profile includes: (a) operating the tuner to thereby adjust the output power signal of the generator from the first energy output profile to a second energy output profile.

### Example 12

The method of any one or more of Examples 1 through 11, wherein the electrical parameter threshold includes an electrical current threshold.

### Example 13

The method of any one or more of Examples 1 through 12, wherein the induced electrical parameter includes an induced electrical current.

### Example 14

The method of any one or more of Examples 1 through 13, wherein the first energy output profile provides a first voltage, wherein the second energy output profile provides a second voltage, wherein the second voltage is lower than the first voltage.

### Example 15

The method of Example 14, wherein the wherein the first energy output profile provides a first power level, wherein the second energy output profile provides a second power level, wherein the second power level is the same as the first power level.

### Example 16

An electrosurgical system, comprising: (a) an instrument, including: (i) a body, (ii) an end effector coupled with a distal end of the body, wherein the end effector includes an electrode operable to apply RF energy to tissue of a patient, and (ii) a conductive component coupled with the body, wherein the conductive component is configured to collect a capacitive coupling current that is induced by application of the RF energy by the electrode; (b) a generator configured to provide the RF energy to the electrode; and (c) a controller operatively coupled with the generator and configured to (i) determine a current threshold of capacitive coupling for monitoring on the conductive component during an operation, (ii) activate the electrode of the instrument by applying an output power signal to the electrode from the generator, (iii) monitor an induced current on the conductive component of the instrument, wherein the induced current includes a parasitic energy loss originating from the electrode, and (iv) when the induced current meets or exceeds the current threshold during the operation, adjust the output power signal of the generator to reduce the induced current until the induced current falls below the current threshold of capacitive coupling while maintaining delivery of energy to the electrode.

### Example 17

The electrosurgical system of Example 16, further comprising a tuner coupled with the generator, wherein the controller is configured to selectively operate the tuner to adjust the output power signal of the generator.

### Example 18

The electrosurgical system of any one or more of Examples 16 through 17, further comprising one or more sensors operatively coupled with the controller and configured to measure the capacitive coupling current and provide a current measurement to the controller.

### Example 19

The electrosurgical system of Example 18, wherein at least one of the one or more sensors is configured to measure an impedance value, wherein the controller is further configured to: (i) determine an impedance change threshold for monitoring during an operation, (ii) monitor for a change in the impedance value, and (iii) when the change of the impedance value meets or exceeds the impedance change threshold during the operation, adjust the output power signal of the generator.

### Example 20

The electrosurgical system of any one or more of Examples 16 through 19, wherein, to adjust the output power signal, the controller is configured to adjust at least one of a voltage magnitude, a current limit, or an power limit.

### Example 21

The electrosurgical system of Example 16, wherein the generator is configured to apply monopolar RF energy to a patient.

### Example 22

The electrosurgical system of Example 21, wherein the monopolar RF energy has a frequency of between approximately 300 kHz and approximately 500 kHz.

### Example 23

An electrosurgical system, comprising: (a) an instrument, including: (i) a body, (ii) an end effector coupled with a distal end of the body, wherein the end effector includes an electrode operable to apply RF energy to tissue of a patient, and (ii) a conductive component coupled with the body, wherein the conductive component is configured to collect a capacitive coupling current that is induced by application of the RF energy by the electrode; (b) a generator configured to provide the RF energy sufficient to cut or seal tissue to the electrode; (c) a sensor configured to measure the capacitive coupling current; and (d) a controller operatively coupled with the generator and the sensor and configured to: (i) determine a current threshold of capacitive coupling for monitoring on the conductive component during an operation, (ii) monitor an induced current on the conductive component of the instrument, and (iii) when the induced current meets or exceeds the current threshold during the operation, adjust the RF energy provided by the generator to reduce the induced current until the induced current falls below the current threshold of capacitive coupling while maintaining delivery of energy to the electrode.

### VI. MISCELLANEOUS

Versions of the devices described above may have application in conventional medical treatments and procedures conducted by a medical professional, as well as application in robotic-assisted medical treatments and procedures.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

In addition to the foregoing, the teachings herein may be readily combined with the teachings of U.S. Pat. App. No. [ATTORNEY DOCKET NO. END9294USNP1.0735554], entitled “Filter for Monopolar Surgical Instrument Energy Path,” filed on even date herewith, the disclosure of which is incorporated by reference herein. Various suitable ways in which the teachings herein may be combined with the teachings of U.S. Pat. App. No. [ATTORNEY DOCKET NO. END9294USNP1.0735554] will be apparent to those of ordinary skill in the art in view of the teachings herein.

In addition to the foregoing, the teachings herein may be readily combined with the teachings of U.S. Pat. App. No. [ATTORNEY DOCKET NO. END9294USNP3.0735558], entitled “Energized Surgical Instrument System with Multi-Generator Output Monitoring,” filed on even date herewith, the disclosure of which is incorporated by reference herein. Various suitable ways in which the teachings herein may be combined with the teachings of U.S. Pat. App. No. [ATTORNEY DOCKET NO. END9294USNP3.0735558] will be apparent to those of ordinary skill in the art in view of the teachings herein.

In addition to the foregoing, the teachings herein may be readily combined with the teachings of U.S. Pat. App. No. [ATTORNEY DOCKET NO. END9294USNP4.0735564], entitled “Electrosurgical Instrument with Shaft Voltage Monitor,” filed on even date herewith, the disclosure of which is incorporated by reference herein. Various suitable ways in which the teachings herein may be combined with the teachings of U.S. Pat. App. No. [ATTORNEY DOCKET NO. END9294USNP4.0735564] will be apparent to those of ordinary skill in the art in view of the teachings herein.

In addition to the foregoing, the teachings herein may be readily combined with the teachings of U.S. Pat. App. No. [ATTORNEY DOCKET NO. END9294USNP5.0735566], entitled “Electrosurgical Instrument with Electrical Resistance Monitor at Rotary Coupling,” filed on even date herewith, the disclosure of which is incorporated by reference herein. Various suitable ways in which the teachings herein may be combined with the teachings of U.S. Pat. App. No. [ATTORNEY DOCKET NO. END9294USNP5.0735566] will be apparent to those of ordinary skill in the art in view of the teachings herein.

In addition to the foregoing, the teachings herein may be readily combined with the teachings of U.S. Pat. App. No. [ATTORNEY DOCKET NO. END9294USNP6.0735568], entitled “Electrosurgical Instrument with Modular Component Contact Monitoring,” filed on even date herewith, the disclosure of which is incorporated by reference herein. Various suitable ways in which the teachings herein may be combined with the teachings of U.S. Pat. App. No. [ATTORNEY DOCKET NO. END9294USNP6.0735568] will be apparent to those of ordinary skill in the art in view of the teachings herein.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method for performing an electrosurgical procedure using an instrument system, wherein the instrument system includes (a) a surgical instrument having an electrode configured to operate on a tissue of a patient, (b) a generator for powering the electrode, and (c) one or more sensors configured to measure electrical energy flowing between the generator and the patient, the method comprising:
(a) determining an electrical parameter threshold of capacitive coupling for monitoring on a conductive component of the surgical instrument during an operation;
(b) activating the electrode of the surgical instrument by applying an output power signal from the generator to the electrode, wherein the output power signal has a first energy output profile;
(c) monitoring an induced electrical parameter on the conductive component of the surgical instrument via the one or more sensors, the induced electrical parameter being associated with the determined electrical parameter threshold, wherein the induced electrical parameter includes a parasitic energy loss; and
(d) when the induced electrical parameter measured from the conductive component of the surgical instrument meets or exceeds the electrical parameter threshold during the operation, adjusting the output power signal of the generator from the first energy output profile to a second energy output profile, wherein the adjustment is operable to reduce the induced electrical parameter measured from the conductive component of the surgical instrument, wherein the adjustment is further operable to reduce the parasitic energy loss without ceasing delivery of energy to the electrode.

2. The method of claim 1, wherein the conductive component of the surgical instrument is configured to avoid coming into contact with the patient during the operation, the conductive component being separate from the electrode.

3. The method of claim 1, wherein a first sensor of the one or more sensors is configured to measure electrical energy communicated from the generator to the patient, wherein a second sensor of the one or more sensors is configured to measure electrical energy communicated from the patient to the generator, wherein the instrument system is configured to measure an impedance of the patient between the first and second sensors, the method further comprising:
(a) determining an impedance change threshold for monitoring during an operation;
(b) monitoring for a change in the impedance of the patient between the first and second sensors; and
(c) when the change of the impedance of the patient meets or exceeds the impedance change threshold during the operation, adjusting the output power signal of the generator from the first energy output profile to the second energy output profile.

4. The method of claim 1, wherein adjusting the output power signal includes adjusting at least one of a voltage magnitude, a current limit, or a power limit.

5. The method of claim 1, further comprising:
(a) upon adjusting the output power signal from the first energy output profile to the second energy output profile, determining whether the generator has reached a power output adjustment limit and is thereby incapable of adjusting the output power signal from the first energy output profile to the second energy output profile; and
(b) if the generator has reached the power adjustment limit, disconnecting the output power signal from the electrode.

6. The method of claim 1, wherein the conductive component of the surgical instrument includes a metallic shield.

7. The method of claim 1, further comprising:
(a) prior to activating the electrode of the surgical instrument, positioning a ground electrode on the patient so as to create a current path in the tissue of the patient between the electrode and the ground electrode, wherein the ground electrode includes an electrical lead coupled with an electrical ground node.

8. The method of claim 1, wherein the generator is configured to apply monopolar RF energy to the patient.

9. The method of claim 1, wherein the surgical instrument is a handheld instrument.

10. The method of claim 1, wherein the surgical instrument is a component of a robotic electrosurgical system.

11. The method of claim 1, wherein the instrument system further includes a tuner coupled with the generator, wherein the tuner is selectively operable to adjust the output power signal of the generator, wherein adjusting the output power signal of the generator from the first energy output profile to a second energy output profile includes:
(a) operating the tuner to thereby adjust the output power signal of the generator from the first energy output profile to a second energy output profile.

12. The method of claim 1, wherein the electrical parameter threshold includes an electrical current threshold.

13. The method of claim 1, wherein the induced electrical parameter includes an induced electrical current.

14. The method of claim 1, wherein the first energy output profile provides a first voltage, wherein the second energy output profile provides a second voltage, wherein the second voltage is lower than the first voltage.

15. The method of claim 14, wherein the wherein the first energy output profile provides a first power level, wherein the second energy output profile provides a second power level, wherein the second power level is the same as the first power level.

16. An electrosurgical system, comprising:
(a) an instrument, including:
(i) a body,
(ii) an end effector coupled with a distal end of the body, wherein the end effector includes an electrode operable to apply RF energy to tissue of a patient, and
(ii) a conductive component coupled with the body, wherein the conductive component is configured to collect a capacitive coupling current that is induced by application of the RF energy by the electrode;
(b) a generator configured to provide the RF energy to the electrode; and
(c) a controller operatively coupled with the generator and configured to:
(i) determine a current threshold of capacitive coupling for monitoring on the conductive component during an operation,
(ii) activate the electrode of the instrument by applying an output power signal to the electrode from the generator,
(iii) monitor an induced current on the conductive component of the instrument, wherein the induced current includes a parasitic energy loss originating from the electrode, and
(iv) when the induced current meets or exceeds the current threshold during the operation, adjust the output power signal of the generator to reduce the induced current until the induced current falls below the current threshold of capacitive coupling while maintaining delivery of energy to the electrode.

17. The electrosurgical system of claim 12, further comprising a tuner coupled with the generator, wherein the controller is configured to selectively operate the tuner to adjust the output power signal of the generator.

17. The electrosurgical system of claim 16, further comprising one or more sensors operatively coupled with the controller and configured to measure the capacitive coupling current and provide a current measurement to the controller.

18. The electrosurgical system of claim 17, wherein at least one of the one or more sensors is configured to measure an impedance value, wherein the controller is further configured to:
(i) determine an impedance change threshold for monitoring during an operation,
(ii) monitor for a change in the impedance value, and
(iii) when the change of the impedance value meets or exceeds the impedance change threshold during the operation, adjust the output power signal of the generator.

19. The electrosurgical system of claim 16, wherein, to adjust the output power signal, the controller is configured to adjust at least one of a voltage magnitude, a current limit, or an power limit.

17. The electrosurgical system of claim 12, wherein the generator is configured to apply monopolar RF energy to a patient.

18. The electrosurgical system of claim 17, wherein the monopolar RF energy has a frequency of between approximately 300 kHz and approximately 500 kHz.

20. An electrosurgical system, comprising:
(a) an instrument, including:
(i) a body,
(ii) an end effector coupled with a distal end of the body, wherein the end effector includes an electrode operable to apply RF energy to tissue of a patient, and
(ii) a conductive component coupled with the body, wherein the conductive component is configured to collect a capacitive coupling current that is induced by application of the RF energy by the electrode;
(b) a generator configured to provide the RF energy sufficient to cut or seal tissue to the electrode;
(c) a sensor configured to measure the capacitive coupling current; and
(d) a controller operatively coupled with the generator and the sensor and configured to:
(i) determine a current threshold of capacitive coupling for monitoring on the conductive component during an operation,
(ii) monitor an induced current on the conductive component of the instrument, and
(iii) when the induced current meets or exceeds the current threshold during the operation, adjust the RF energy provided by the generator to reduce the induced current until the induced current falls below the current threshold of capacitive coupling while maintaining delivery of energy to the electrode.
